(19) United States
(12) Patent Application Publication
(10) Pub. No.: US 2015/0258198 A1
(43) Pub. Date: Sep. 17, 2015

(54) Pharmaceutical microparticulate compositions of polypeptides

(71) Applicant: WOCKHARDT LIMITED, Aurangabad 0, (IN)

(72) Inventors: Mukesh Kumar, Delhi 5 (IN); Girish Kumar Jain, Delhi 4 (IN)

(21) Appl. No.: 14/425,233

(22) PCT Filed: May 2, 2013
(86) PCT No.: PCT/IB2013/053473

§ 371 Date: Mar. 2, 2015

(30) Foreign Application Priority Data
Aug. 14, 2012 (IN) 2369/MUM/2012
Aug. 14, 2012 (IN) 2370/MUM/2012

(51) Int. Cl. A61K 47/38; A61K 47/26; A61K 47/36; A61K 38/22; A61K 45/06

(52) U.S. Cl. CPC A61K 47/38; A61K 38/2278; A61K 45/06; A61K 47/36; A61K 47/26

(57) ABSTRACT

The present invention refers to pharmaceutical microparticulate compositions of biologically active polypeptides and methods of forming and using such compositions. More particularly, microparticle compositions for sustained release of biologically active polypeptides are provided. By using particular pharmaceutical excipients or their specific quantity, compositions of biologically active polypeptides with desired release profile can be prepared. The microparticle compositions of this invention comprise a biocompatible polymer, a biologically active polypeptide, and pharmaceutical excipients.

### FIELD OF THE INVENTION

The present invention relates to pharmaceutical microparticulate compositions of biologically active polypeptides, and methods of forming and using such compositions. More particularly, the present invention relates to microparticle compositions of biologically active polypeptides using particular pharmaceutical excipients.

### BACKGROUND OF THE INVENTION

Numerous proteins and peptides, collectively referred to herein as polypeptides, exhibit biological activity in vivo and are useful as medicaments. Many illnesses or conditions require administration of a sustained level of medicament to provide the most effective prophylactic and/or therapeutic effects. Sustained levels are often achieved by the administration of biologically active polypeptides by frequent subcutaneous injections, which often result in fluctuating levels of medicament and poor patient compliance.

As an alternative, the use of biodegradable materials, such as polymers, encapsulating the medicament can be employed as a sustained delivery system. The use of biodegradable polymers, for example, in the form of microparticles or microcarriers, can provide a sustained release of medicament, by utilizing the inherent biodegradability of the polymer to control the release of the medicament thereby providing a more consistent, sustained level of medicament and improved patient compliance.

However, these sustained release compositions can often exhibit high initial bursts of medicament and minimal release thereafter, resulting in serum drug levels outside the therapeutic window and/or poor bioavailability of the medicament. In addition, the presence of polymer, physiological temperatures and body response to the sustained release composition can cause the medicament to be altered (e.g., degraded, aggregated) thereby interfering with the desired release profile for the medicament.

Further, methods used to form sustained release microparticle compositions can result in loss of activity of the medicament due to the instability of the medicament and the degradative effects of the processing steps. Degradative effects are particularly problematic when the medicament is a polypeptide.

Several attempts have been made earlier to prepare sustained release microparticle compositions of polypeptides in order to achieve desirable and consistent release profile.

U.S. Pat. Nos. 5,654,008, 5,945,126 and 6,194,006 discloses various methods for preparing microparticles.

U.S. Pat. No. 7,456,254 discloses a composition for the sustained-release of biologically active polypeptides containing a dispersion of biologically active polypeptides dispersed in a biocompatible polymer and sucrose.

U.S. Pat. No. 7,164,005 discloses a composition for the sustained release of a biologically active polypeptides containing a dispersion of biologically active polypeptides dispersed in a biocompatible polymer, a sugar, a salting-out salt, and a corticosteroid.

U.S. Pat. No. 6,558,702 discloses a sustained release composition comprising a biocompatible polymer having a biologically active protein, peptide or nucleic acid incorporated therein, and a bisphosphonate compound present in an amount sufficient to modify the release profile of the biologically active protein, peptide or nucleic acid from the sustained release composition.

U.S. Patent Application No. 20040208929 discloses a composition for the sustained release of biologically active polypeptide comprising a biocompatible polymer having dispersed therein a biologically active polypeptide, a sugar and glycine.

It can be evident from the prior art that use of sugar was suggested for preparing sustained release compositions of biological active peptides in order to increase the drug bioavailability or improve its release profile from the composition.

Further, incorporation of additional ingredients can alter the rate of release of biologically active peptides from the composition in an attempt to modify the release profile or improve drug retaining capacity of the microparticles.

Therefore, a need exists for alternate compositions of biologically active polypeptides which not only exhibits advantages of employing sugar in the composition, deliver the polypeptide in therapeutic levels with a desired release rate but also retains activity and potency for the polypeptide over desired period.

While much work has been developed that addresses aforesaid problems, novel solutions are required, and despite of the various compositions available, it still remains challenging to develop simple microparticle compositions of biologically active peptides, which can serve the aforesaid objectives.

### SUMMARY OF THE INVENTION

In one aspect, the present invention provides a microparticulate composition for sustained release of one or more biologically active polypeptides comprising one or more biocompatible polymers, one or more natural or synthetic mucilages, one or more sugars and optionally, one or more pharmaceutical excipients.

In another aspect, the present invention provides a microparticulate composition for sustained release of one or more biologically active polypeptides comprising one or more biocompatible polymers, one or more corticosteroids, one or more natural or synthetic mucilages and optionally, one or more pharmaceutical excipients.

In another aspect, the present invention provides a microparticulate composition for sustained release of exendin-4 or agonists, analogs or derivatives thereof comprising one or more biocompatible polymer, one or more natural or synthetic mucilages, one or more sugars and optionally, one or more pharmaceutical excipients.

In another aspect, the present invention provides a microparticulate composition for sustained release of one or more biologically active polypeptides comprising one or more biocompatible polymers, one or more natural or synthetic mucilages, one or more sugars and optionally, one or more pharmaceutical excipients, wherein the composition is devoid of any salting out salts.

In another aspect, the present invention provides a microparticulate composition for sustained release of one or more biologically active polypeptide comprising one or more biocompatible polymers, and optionally, one or more pharmaceutical excipients, wherein the composition comprises more than 5% w/w of one or more sugars.

In another aspect, the present invention provides a microparticulate composition for sustained release of one or more biologically active polypeptides comprising one or more biocompatible polymers, one or more natural or synthetic mucilages, one or more sugars and optionally, one or more pharmaceutical excipients, wherein the composition comprises more than 5% w/w of one or more sugars.

In another aspect, the present invention provides a microparticulate composition for sustained release of one or more biologically active polypeptide comprising one or more biocompatible polymers, one or more corticosteroids and optionally, one or more pharmaceutical excipients, wherein the composition comprises more than 5% w/w of one or more sugars.

In another aspect, the present invention provides a microparticulate composition for sustained release of exendin-4 or agonists, analogs or derivatives thereof comprising one or more biocompatible polymer and optionally, one or more pharmaceutical excipients, analogs or derivatives thereof; wherein the composition comprises more than 5% w/w of one or more sugars.

In another aspect, the present invention provides a microparticulate composition for sustained release of one or more biologically active polypeptide comprising one or more biocompatible polymers, one or more a biologically active polypeptide, more than 5% w/w of one or more sugars and optionally, one or more pharmaceutical excipients, wherein the composition is free of salting out salts.

In another aspect, the present invention provides a microparticulate composition for sustained release of exenatide consisting essentially of one or more biocompatible polymers, gelatin, sucrose and optionally, one or more pharmaceutical excipients.

In another aspect, the present invention provides a process for preparing the microparticles of biologically active polypeptide comprises: preparing an emulsion that comprises a first phase and a second phase, the first phase comprising biologically active polypeptide, one or more natural or synthetic mucilages, one or more sugars and optionally, one or more pharmaceutical excipients; the second phase comprising one or more biodegradable polymers and one or more solvents for the polymer; quenching and washing of the emulsion to form microparticle suspension; and optionally, lyophilizing the microparticle suspension to form polypeptide containing microparticles.

In another aspect, the present invention provides a process for preparing the microparticles of biologically active polypeptides comprises: preparing an emulsion that comprises a first phase and a second phase, the first phase comprising biologically active polypeptide, more than 5% w/w of one or more sugars and optionally, one or more pharmaceutical excipients, the second phase comprising one or more biodegradable polymers and one or more solvents for the polymer; quenching and washing of the emulsion to form microparticle suspension; and optionally, lyophilizing the microparticle suspension to form polypeptide containing microparticles.

In another aspect, the present invention provides a method of treating a patient having Type 2 diabetes comprises of: administering a microparticulate composition comprising one or more biocompatible polymers, one or more natural or synthetic mucilages, one or more sugars and optionally, one or more pharmaceutical excipients.

In another aspect, the present invention provides a method of treating a patient having Type 2 diabetes comprises of: administering a microparticulate composition comprising one or more biocompatible polymers, and one or more a biologically active polypeptide, and optionally, one or more pharmaceutical excipients, wherein the composition comprises more than 5% w/w of one or more sugars.

Embodiments of the pharmaceutical composition may include one or more of the following features. For example, the pharmaceutically acceptable excipients may include solubilizers, anti-oxidants, buffering agents, pH adjusting agents, co-solvents, chelating agents, stabilizers, preservatives, lubricants, tonicity adjusting agents, cryoprotectants and the like known to the art used either alone or in combination thereof.

### DETAILED DESCRIPTION OF THE INVENTION

The present invention relates to novel microparticle compositions of biologically active polypeptides and methods of forming and using such compositions.

The inventors of the present invention have surprisingly found that it is possible to formulate microparticle compositions of biologically active polypeptides exhibiting desired release profiles. The microparticulate compositions with a desirable and consistent release profile can be achieved with formulations containing natural or synthetic mucilages and/or high molecular weight compounds or by using more than 5% w/w of sugar by total weight of the composition.

It was also found that by using particular excipients, leaching of the polypeptides from the microparticles can be reduced significantly, particularly when the manufacturing process of the microparticles involve water as an aqueous phase or when water is used as vehicle for reconstitution prior to administration of the microparticles. The resulting microparticles may also exhibit improved polypeptide loading and entrapment ability.

Further, by using more than 5% w/w of sugar, optionally with other pharmaceutical excipients, microparticles with high polypeptides entrapment and retention capacity can be produced. The resulting microparticles may also exhibit excellent physical and/or chemical stability.

The inventors of the present invention further found that loss of activity of the biologically active polypeptide due to instability and/or chemical interactions between the biologically active polypeptide and other components, which are contained in or used in formulating the sustained release composition, can also be minimized.

The bioavailability of polypeptide in the microparticulate composition can be increased by using particular pharmaceutical excipients in accordance with the present invention over compositions lacking the specific combination of excipients resulting in sustained release composition of the invention, which can deliver therapeutic levels of drug for desired period. Most notably, microparticle compositions having this specific and optimized amount of excipients can exhibit at least equivalent bioavailability of the polypeptide over compositions containing sugars alone, thereby resulting in an sustained release composition which can deliver therapeutic levels of polypeptide for a desired period.

In addition, the microparticulate compositions of the present invention may also exhibit a reduced lag phase, which can provide for a smoothing out of the release profile and can also contribute to an increase in the amount of polypeptide released if desired.

Further advantages of the microparticulate formulation for biologically active polypeptide as described herein include increased patient compliance and acceptance by eliminating the need for repetitive administration, increased therapeutic benefit by eliminating fluctuations in active agent concentration in blood levels by providing a desirable release profile, and a potential lowering of the total amount of biologically active polypeptide necessary to provide a therapeutic benefit by reducing these fluctuations.

The composition of the present invention comprises of, microparticles comprising a biocompatible polymer, a biologically active polypeptide, one or more natural or synthetic mucilages, one or more sugars and optionally, one or more pharmaceutical excipients. Preferably, the composition comprises more than 5% w/w of sugar.

In an embodiment, the microparticles of the composition of the present invention comprises a biocompatible polymer, a biologically active polypeptide, one or more natural or synthetic mucilages, and optionally one or more pharmaceutical excipients, which do not alter the rate of release of the biologically active polypeptide from the microparticles.

In a further embodiment, the microparticles of the composition of the present invention comprises a biocompatible polymer, a biologically active polypeptide, more than 5% w/w of sugar and one or more pharmaceutical excipients, which do not alter the rate of release of the biologically active polypeptide from the microparticles.

The term “microparticles” or “microparticulate” as used herein refers to solid particles that contain a biologically active polypeptide dispersed or dissolved within a polymer that serves as the matrix of the particle. The polymer is preferably biodegradable and biocompatible.

Biologically active polypeptides as used herein collectively refers to biologically active proteins and peptides and the pharmaceutically acceptable salts thereof, which are in their molecular, biologically active form when released in vivo, thereby possessing the desired therapeutic, prophylactic and/or diagnostic properties in vivo. Typically, the polypeptide has a molecular weight between 500 and 200,000 Daltons.

Suitable biologically active polypeptides include, but are not limited to, glucagon, glucagon-like peptides such as, GLP-1, GLP-2 or other GLP analogs, derivatives or agonists of Glucagon Like Peptides, exendins such as, exendin-3 and exendin-4, derivatives, agonists and analogs thereof, vasoactive intestinal peptide (VIP), immunoglobulins, antibodies, cytokines (e.g., lymphokines, monokines, chemokines), interleukins, macrophage activating factors, interferons, erythropoietin, nucleases, tumor necrosis factor, colony stimulating factors (e.g., G-CSF), insulin, enzymes (e.g., superoxide dismutase, plasminogen activator, etc.), tumor suppressors, blood proteins, hormones and hormone analogs and agonists (e.g., follicle stimulating hormone, growth hormone, adrenocorticotropic hormone, and luteinizing hormone releasing hormone (LHRH)), vaccines (e.g., tumoral, bacterial and viral antigens), antigens, blood coagulation factors, growth factors (NGF and EGF), gastrin, GRH, antibacterial peptides such as defensin, enkephalins, bradykinins, calcitonin and muteins, analogs, truncation, deletion and substitution variants and pharmaceutically acceptable salts of all the foregoing.

Suitable biologically active polypeptide for the purpose of the present inventions comprises one or more antidiabetic or glucoregulatory polypeptides, including GLP-1, GLP-2, exendin-3, exendin-4 or an analog, derivative or agonist thereof. The preferred polypeptide is exendin-4. However, it could be possible to use other polypetides to devise sustained release formulations with aforesaid objectives of the present invention.

Exendin-4 is a 39 amino acid polypeptide. Exendin-4 has been shown in humans and animals to stimulate secretion of insulin in the presence of elevated blood glucose concentrations, but not during periods of low blood glucose concentrations (hypoglycemia). It has also been shown to suppress glucagon secretion, slow gastric emptying and affect food intake and body weight, as well as other actions. As such, exendin-4 and analogs and agonists thereof can be useful in the treatment of diabetes mellitus, IGT, obesity, etc.

The amount of biologically active polypeptide, which is contained within the polymeric matrix of a sustained release composition, is a therapeutically, diagnostically or prophylactically effective amount which can be determined by a person of ordinary skill in the art, taking into consideration factors such as body weight, condition to be treated, type of polymer used, and release rate from the polymer.

Preferably, the microparticle composition of the present invention contains from about 0.01% w/w to about 50% w/w of the biologically active polypeptide (such as exendin-4) by total weight of composition. For example, the amount of biologically active polypeptide (such as exendin-4) can be from about 0.1% w/w to about 30% w/w by total weight of the composition. The amount of polypeptide will vary depending upon the desired effect, potency of the agent, the planned release levels, and the time span over which the polypeptide will be released. Preferably, the range of loading is between about 0.1% w/w to about 10% w/w, for example, 0.5% w/w to about 5% w/w. Desired release profiles may be obtained when the biologically active polypeptide, e.g. exendin-4, is loaded at about 1% w/w to about 10% w/w.

In a particular embodiment, the microparticle composition comprises a biocompatible polymer, an antidiabetic or glucoregulatory polypeptide, one or more natural or synthetic mucilages and optionally one or more pharmaceutical excipients. More specifically, the polypeptide is selected from GLP-1, GLP-2, exendin-3, exendin-4 or an analog, derivative or agonist thereof. Preferably, the polypeptide is exendin-4, and more preferably, the polypeptide is exenatide.

The use of a particular pharmaceutical excipient or its specific quantity in the microparticle compositions of the invention may improve the bioavailability of the incorporated biologically active polypeptide, e.g, anti-diabetic or glucoregulatory peptides, and minimizes loss of activity due to instability and/or chemical interactions between the polypeptide and other components contained or used in formulating the microparticle composition, while maintaining an excellent release profile.

The sugar used in the composition of the present invention is a monosaccharide, disaccharide or oligosaccharide (from about 3 to about 10 monosaccharides) or a derivative thereof. For example, sugar alcohols of monosaccharides are suitable derivatives included in the present definition of sugar. As such, the sugar alcohol mannitol, for example, which is derived from the monosaccharide mannose is included in the definition of sugar as used herein.

Suitable monosaccharides include, but are not limited to, glucose, fructose and mannose. A disaccharide, as further defined herein, is a compound which upon hydrolysis yields two molecules of a monosaccharide. Suitable disaccharides include, but are not limited to, sucrose, lactose and trehalose. Suitable oligosaccharides include, but are not limited to, raffinose and acarbose. The preferred sugar is selected from sucrose, mannitol, lactose, glucose, fructose or mixture thereof.

The amount of sugar in the microparticle composition of the present invention may range between about 1% w/w to about 30% w/w. Preferably; microparticle composition of the present invention contains more than 5% w/w of sugar. The more preferred amount of sugar ranges between about 5% w/w to about 30% w/w. Excellent release profiles were obtained incorporating about 10% w/w sugar.

Alternatively, the amount of sugar present in the microparticle composition can be referred to on a weight ratio with the agent or biologically active polypeptide. For example, the polypeptide and sugar can be present in a ratio from about 10:1 to about 1:10 w/w. In a particularly preferred embodiment, the ratio of polypeptide (e.g., exendin-4) to sugar (e.g., sucrose) is about 3:2 (w/w).

Polymers suitable to form the microparticles of the present invention are biocompatible polymers, which can be either biodegradable or non-biodegradable polymers or blends or copolymers thereof. A polymer is biocompatible if the polymer and any degradation products of the polymer are non-toxic to the recipient and also possess no significant deleterious or untoward effects on the recipient's body, such as a substantial immunological reaction at the injection site.

Biodegradable, as defined herein, means the composition will degrade or erode in vivo to form smaller units or chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl flouride, poly(vinyl imidazole), chlorosulphonate polyolefins, polyethylene oxide, blends thereof, and copolymers thereof.

Acceptable molecular weights for polymers used in the invention can be determined by a person of ordinary skill in the art taking into consideration factors such as the desired polymer degradation rate, physical properties such as mechanical strength, end group chemistry and rate of dissolution of polymer in solvent. Typically, an acceptable range of molecular weight is of about 2,000 Daltons to about 2,000,000 Daltons. In a preferred embodiment, the polymer is biodegradable polymer or copolymer. In a more preferred embodiment, the polymer is a poly(lactide-co-glycolide) (hereinafter “PLG”) with a lactide:glycolide ratio of about 1:1 and a molecular weight of about 10,000 Daltons to about 90,000 Daltons. In an even more preferred embodiment, the molecular weight of the PLG used in the present invention has a molecular weight of about 30,000 Daltons to about 70,000 Daltons such as about 50,000 to about 60,000 Daltons.

The PLGs can possess acid end groups or blocked end groups, such as can be obtained by esterifying the acid. Excellent results were obtained with a PLG with an acid end group.

Polymers can also be selected based upon the polymer's inherent viscosity. Suitable inherent viscosities include about 0.06 to 1.0 dL/g, such as about 0.2 to 0.6 dL/g, more preferably between about 0.3 to 0.5 dL/g. Preferred polymers are chosen that will degrade in 3 to 4 weeks.

The natural or synthetic mucilages of the microparticle composition of the present invention may not alter the release profile or contribute in providing desired release of the polypeptide from the composition.

The natural or synthetic mucilages suitable for present invention can be selected from, one or more of gum acacia, Irish moss, gum karaya, gum tragacanth, gum guaiac, gum xanthane, locust bean gum, etc., while natural high molecular weight compounds include, among others, various proteins such as casein, gelatin, collagen, albumin (e.g. human serum albumin), globulin, fibrin, etc. and various carbohydrates such as cellulose, dextrin, pectin, starch, agar, mannan, etc. These substances may be used as they are or in chemically modified forms, e.g. esterified or etherified forms (e.g. methylcellulose, ethylcellulose, carboxymethylcellulose, gelatin succinate, etc.), hydrolyzed forms (e.g. sodium alginate, sodium pectinate, etc.) or salts thereof. The amount of these compounds in the microparticle composition of the present invention may range from about 0.05% w/w to about 80% w/w by weight of the composition.

Among the aforementioned compounds, gelatin, albumin, pectin and agar are particularly preferred.

The microparticle composition of the invention may further comprise additional excipients, which may not interfere the desired release profile of the biologically active polypeptide. It is also possible to select additional excipient in order to contribute the overall desired release profile of polypeptide in the composition of the present invention.

Such additional excipients can be used in the composition to further increase or decrease the rate of release of the polypeptide to desired level.

Ingredients which can substantially increase the rate of release include pore forming agents and excipients which facilitate polymer degradation. For example, the rate of polymer hydrolysis is increased in non-neutral pH. Therefore, an acidic or a basic excipient such as an inorganic acid or inorganic base can be added to the polymer solution, used to form the microparticles, to alter the polymer erosion rate. Ingredients which can substantially decrease the rate of release include excipients that decrease the water solubility of the agent.

In an embodiment of the described microparticle compositions consists essentially of the biocompatible polymer, a biologically active polypeptide, a natural or synthetic mucilages, one or more sugars and optionally, one or more pharmaceutical excipients, wherein the composition is free of excipients which increase or decrease the rate of release of the biologically active polypeptide from the microparticles.

In a further embodiment, the microparticle compositions consists essentially of the biocompatible polymer, a biologically active polypeptide, more than 5% w/w of sugar and one or more pharmaceutical excipients, wherein the composition is free of excipients, which increase or decrease the rate of release of the biologically active polypeptide from the microparticles.

In a further embodiment, the microparticle compositions consists essentially of the biocompatible polymer, a biologically active polypeptide, a natural or synthetic mucilages, one or more sugars and optionally, one or more pharmaceutical excipients, wherein the composition comprises more than 5% w/w of sugar and is free of excipients which increase or decrease the rate of release of the biologically active polypeptide from the microparticles.

By “consists essentially of” is meant the absence of ingredients which substantially increase the rate of release of the biologically active polypeptide from the formulation. Examples of additional excipients which would not be expected to substantially increase or decrease the rate of release of the biologically active polypeptide include additional active agents and inert ingredients.

In yet another embodiment, the microparticles consists of the biocompatible polymer, a biologically active polypeptide and the gelatin.

In a still further embodiment, the microparticles consists of the biocompatible polymer, a biologically active polypeptide, more than 5% w/w of sugar and one or more pharmaceutical excipients.

By “consists of” is meant the absence of components or ingredients other than those listed and residual levels of starting materials, solvents, etc. from the process.

It has been, further surprisingly found that sugar and buffering agents such as acetate, citrate, phosphate or other biologically compatible buffer may not be necessary in the aqueous phase to achieve a sustained release formulation of polypeptide, such as, exendin-4 (e.g. exenatide), with good to excellent bioavailability. As such, the compositions of the invention also include microparticles, as described herein, in the substantial (or complete) absence of buffer and/or salting out salts.

In an embodiment, the microparticles of the composition of the present invention comprises a biocompatible polymer, a biologically active polypeptide, one or more natural or synthetic mucilages, one or more sugars and optionally one or more pharmaceutical excipients, wherein the composition is free of salting out salts.

The sustained release composition of the present invention comprises plurality of microparticles loaded with biologically active polypeptides. A microparticle, as defined herein, comprises a polymer component having a diameter of less than about one millimeter and having biologically active polypeptide dispersed or dissolved therein. A microparticle can have a spherical, non-spherical or irregular shape. Typically, the microparticle will be of a size suitable for injection. A typical size range for microparticles is 1000 microns or less. In a particular embodiment, the microparticle ranges from about one to about 180 microns in diameter.

The compositions of the invention can be administered according to methods generally known in the art. The composition of this invention can be administered to a patient (e.g., a human in need of the agent) or other animal, by injection, implantation (e.g., subcutaneously, intramuscularly, intraperitoneally, intracranially, and intradermally), administration to mucosal membranes (e.g., intranasally, intravaginally, intrapulmonary), or in situ delivery (e.g., by enema or aerosol spray).

The composition can be administered using any dosing schedule which achieves the desired therapeutic levels for the desired period of time. For example, the microparticle composition can be administered and the patient monitored until levels of the drug being delivered return to baseline. Following a return to baseline, the sustained release composition can be administered again. Alternatively, the subsequent administration of the microparticle composition can occur prior to achieving baseline levels in the patient.

For example, when the microparticle has incorporated therein a hormone, particularly an anti-diabetic or glucoregulatory peptide, for example, GLP-1, GLP-2, exendin-3, exendin-4 or agonists, analogs or derivatives thereof, the composition is administered in a therapeutically effective amount to treat a patient suffering from diabetes mellitus, IGT, obesity, cardiovascular (CV) disorder or any other disorder that can be treated by one of the above polypeptides or derivatives, analogs or agonists thereof.

Other conditions which can be treated by administering the microparticle composition of the invention include Type I and Type II diabetes which can be treated with a microparticle composition having insulin incorporated therein. In addition, when the incorporated polypeptide is FSH or analogs thereof the microparticle composition can be used to treat infertility. In other instances, the microparticle composition can be used to treat Multiple Sclerosis when the incorporated polypeptide is beta interferon or a mutein thereof. As can be realized, the microparticle composition can be used to treat disease, which responds to administration of a given polypeptide.

In an embodiment, a method of treating a patient having Type 2 diabetes is provided. The method comprises administering a therapeutically effective amount of a composition comprising, plurality of microparticles comprising a biocompatible polymer having dispersed therein exendin-4 or agonists, analogs or derivatives thereof, one or more natural or synthetic mucilages, one or more sugars and optionally, one or more pharmaceutical excipients.

In another embodiment, the composition of the present invention provides a therapeutically effective blood level of biologically active polypeptide, in a patient for a sustained period by administering to the patient a dose of the microparticle composition described herein.

In a further embodiment, the composition of the present invention may be coadministered with a corticosteroid. Coadministration of the microparticle composition of the invention with a corticosteroid can further increase the bioavailability of the biologically active polypeptide of the composition to desired level.

Corticosteroids, as defined herein, refer to steroidal anti-inflammatory agents also referred to as glucocorticoids.

Suitable corticosteroids include, but are not limited to, 21-Acetoxypregnenolone, Alclometasone, Algestone, Amcinonide, Beclomethasone, Betamethasone, Budesonide, Chloroprednisone, Clobetasol, Clobetasone, Clocortolone, Cloprednol, Corticosterone, Cortisone, Cortivazol, Deflazacort, Desonide, Desoximetasone, Dexamethasone, Disflorasone, Diflucortolone, Difluprednate, Enoxolone, Fluazacort, Flucloronide, Flumethasone, Flunisolide, Flucinolone Acetonide, Fluocinonide, Fluocortin Butyl, Flucortolone, Fluorometholone, Fluperolone Acetate, Fluprednidene Acetate, Fluprednisolone, Flurandrenolide, Fluticasone Propionate, Formocortal, Halcinonide, Halobetasol Propionate, Halometasone, Halopredone Acetate, Hydrocortamate, Hydrocortisone, Loteprednol Etabonate, Mazipredone, Medrysone, Meprednisone, Methylprednisolone, Mometasone Furoate, Paramethasone, Prednicarbate, Prednisolone, Prednisolone 25-Diethylamino-acetate, Prednisolone Sodium Phosphate, Prednisone, Prednival, Prednylidene, Rimexolone, Tixocortol, Triamcinolone (all forms), for example, Triamcinolone Acetonide, Triamcinolone Acetonide 21-oic acid methyl ester, Triamcinolone Benetonide, Triamcinolone Hexacetonide, Triamcinolone Diacetate, pharmaceutically acceptable mixtures thereof and salts thereof and any other derivative and analog thereof.

In one embodiment, the corticosteroid can be co-incorporated into the microparticle comprising the biocompatible polymer and the biologically active polypeptide agent incorporated therein.

In another embodiment, the corticosteroid can be separately incorporated into a second biocompatible polymer. The second biocompatible polymer can be the same or different from the first biocompatible polymer which has the biologically active polypeptide agent incorporated therein.

In yet another embodiment, the corticosteroid can be present in an unencapsulated state but commingled with the microparticles (e.g. embedded in the microparticle shell). For example, the corticosteroid can be solubilized in the vehicle used to deliver the composition. Alternatively, the corticosteroid can be present as a solid suspended in an appropriate vehicle. Further, the corticosteroid can be present as a powder that is commingled with the sustained release composition.

It is understood that the corticosteroid may present in an amount sufficient to increase the bioavailability of the biologically active polypeptide from the composition. Increased bioavailability refers to an increase in the bioavailability of the biologically active polypeptide from the composition when co-administered with a corticosteroid in comparison to the administration in the absence of corticosteroid over a time period beginning at two days post administration and ending at the end of the release cycle for the particular formulation.

As used herein, patient refers to a human, such as a human in need of the agent or therapy, prophylaxis or diagnostic method.

As used herein, the term “sustained release of biologically active polypeptide” is a release of the polypeptide from the composition of the invention which occurs over a period which is longer than that period during which a biologically significant amount of the polypeptide would be available following direct administration of a solution of the polypeptide. It is preferred that a sustained release be a release which occurs over a period of at least about one week, such as at least about two weeks, at least about three weeks or at least about four weeks.

In an embodiment, the sustained release of biologically active polypeptide from the composition of the present invention occur over a period of at least two weeks or at least four weeks.

As used herein, a therapeutically effective amount, prophylactically effective amount or diagnostically effective amount is the amount of the composition needed to elicit the desired biological response following administration.

The term “bioavailability” as used herein, refers to the rate and extent to which the polypeptide reaches the circulation system. Bioavailability can be defined as the calculated Area Under the Curve (AUC) for the release profile of a particular polypeptide during the time period starting at post administration and ending at a predetermined time point. As is understood in the art, the release profile is generated by graphing the serum levels of a biologically active agent in a subject (Y-axis) at predetermined time points (X-axis). Bioavailability is often referred to in terms of % bioavailability, which is the bioavailability achieved for a particular polypeptide following administration of a composition divided by the bioavailability achieved for a particular polypeptide following intravenous administration of the same dose of drug, multiplied by 100.

The release profile of the composition of the present invention can be confirmed by appropriate pharmacokinetic monitoring of the patient's serum for the presence of the biologically active polypeptide agent. For example, specific antibody-based testing (e.g., ELISA and IRMA), as is well known in the art, can be used to determine the concentration of certain biologically active polypeptide agents in the patient's serum.

Pharmacodynamic monitoring of the patient to monitor the therapeutic effects of the biologically active polypeptide agents upon the patient can be used to confirm retention of the biological activity of the released agent. Methods of monitoring pharmacodynamic effects can be selected based upon the biologically active polypeptide agent being administered using widely available techniques.

A number of methods are known by which microparticles in the compositions of the invention can be formed. In many of these processes, the material to be encapsulated is dispersed in a solvent containing a wall forming material (e.g., biocompatible polymer). At a single stage of the process, solvent is removed and thereafter the microparticle product is obtained.

In an embodiment, the process of preparing the microparticles of biologically active polypeptide includes dissolving a biocompatible polymer in a polymer solvent to form a polymer solution, and combining a biologically active polypeptide, one or more natural or synthetic mucilages, one or more sugars, and optionally one or more pharmaceutical excipients with the polymer solution. The biologically active polypeptide, natural or synthetic mucilages, sugars and pharmaceutical excipients can be combined with the polymer solution either alone or in a premixed form.

In another embodiment, the process of preparing the microparticles of biologically active polypeptide includes dissolving a biocompatible polymer in a polymer solvent to form a polymer solution, and combining a biologically active polypeptide, more than 5% w/w of one or more sugars, and one or more pharmaceutical excipients with the polymer solution. The biologically active polypeptide, sugar and pharmaceutical excipients can be combined with the polymer solution either alone or in a premixed form.

The biologically active polypeptide, natural or synthetic mucilages, sugars and pharmaceutical excipients can be combined with the polymer solution either as solids, liquids or suspensions. It is understood that the method of combining the polymer, active and excipients can be performed in any order.

In another embodiment, the process for preparing microparticles comprises: preparing an emulsion that comprises a first phase and a second phase, the first phase comprising biologically active polypeptide, one or more natural or synthetic mucilages, one or more sugars, and optionally one or more pharmaceutical excipients, the second phase comprising one or more biodegradable polymers and one or more solvents for the polymer; quenching and washing of the emulsion to form microparticle suspension; and lyophilizing the microparticle suspension to form drug containing microparticles. Lyophilization of the microparticles is of particular advantage as process may become simple, robust and also require relatively less control of processing parameters. The resulting microparticles prepared using lyophilization processing step may posses excellent shape uniformity, exhibiting good flowability during vial filling and passage through needle at the time of administration.

In a further embodiment, the process of preparing the microparticles includes forming a mixture by combining an aqueous phase comprising water, a biologically active polypeptide, natural or synthetic mucilages, sugar, and one or more pharmaceutical excipients with an oil phase comprising a biocompatible polymer and a solvent for the polymer; forming a water-in-oil emulsion by, for example, sonicating or homogenizing, the mixture; adding an oil to the mixture to form microparticles; transferring the microparticles to a quench solvent to harden the microparticles; collecting the hardened microparticles; and drying the hardened microparticles.

Preferably, the polymer can be present in the oil phase in a concentration ranging from about 3% w/w to about 25% w/w, preferably, from about 4% w/w to about 15% w/w, such as from about 5% w/w to about 10% w/w by weight of the composition. Excellent results were obtained herein using a 1% w/w to about 10% w/w concentration of PLG in the oil phase.

The polymer is generally combined with a polymer solvent. Where the polymer is a PLG, such as those preferred herein, the polymer is added to a solvent for PLG. Such solvents are well known in the art. A preferred solvent is dichloromethane.

The biologically active polypeptide and natural or synthetic mucilages (e.g. gelatin) are added in the aqueous phase, preferably in the same aqueous phase. The concentration of biologically active polypeptide is preferably 10 to 100 mg/g, preferably between 50 to 100 mg/g. The concentration of pharmaceutical excipients is preferably 10 to 50 mg/g and 30 to 50 mg/g.

The two phases are then mixed to form an emulsion. It is preferred that the emulsion be formed such that the inner emulsion droplet size is less than about 1 micron, preferably less than about 0.7 microns, more preferably less than about 0.5 microns, such as about 0.4 microns. Sonicators and homogenizers can be used to form such an emulsion.

A coacervation agent as used herein refers to any oil in which the polymer solution (polymer and solvent) is not readily solubilized into and thereby forms a distinct phase with the polymer solution. Suitable coacervation agents for use in the present invention include, but are not limited to, silicone oil, vegetable oil and mineral oil. In a particular embodiment, the coacervation agent is silicone oil and is added in an amount sufficient to achieve a silicone oil to polymer solvent ratio from about 0.75:1 to about 2:1. In a particular embodiment, the ratio of silicone oil to polymer is from about 1:1 to about 1.5:1. In a preferred embodiment, the ratio of silicone oil to polymer is about 1.5:1.

The resulting mixture is added to a quench, which comprises a polymer non-solvent. Polymer non-solvents are generally well known in the art. A particularly preferred quench comprises a heptane/ethanol solvent system.

Solid polypeptides can also be encapsulated in microparticles using a modified version of the process described above. This modified process can be referred to as a solid/oil/oil (S/O/O).

For example, solid exendin-4 or its analog (e.g. exenatide) was suspended in dichloromethane containing 6% PLG and sonicated for about four minutes on ice. Subsequent processing was conducted in a manner analogous to the W/O/O method.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example: 1

### Process of Preparing the Exenatide-Loaded Microparticles

(A) Inner Water-in-Oil Emulsion Formation:

A water phase containing exenatide was prepared by dissolving exenatide (about 7.5% w/w), sucrose (about 2.6% w/w) and gelatin (about 1.5% w/w) in water. Separately, an oil phase of polymer (about 6% w/w) was prepared by dissolving PLGA polymer in dichloromethane. A water-in-oil emulsion was created by adding water phase in to the oil phase with the aid of a homogenizer or sonication or any other known method (W/O ratio of about 1:23). The resulting coarse emulsion was homogenized at approximately 10,000 rpm at ambient temperature. This resulted in an inner emulsion droplet size of less than 1 micron.

(B) Coacervate Formation:

Silicone oil was then added to the inner emulsion prepared in step A for coacervation. The preferred ratio of silicone oil to dichloromethane is 1.5:1. During the coacervation, dichloromethane from the polymer solution partitions into the silicone oil and begins to precipitate the polymer around the water phase containing exenatide, leading to microencapsulation.

(C) Microparticle Hardening/Quenching and Rinse:

Microparticles were added to a heptane/ethanol solvent mixture of 9:1 ratio in a contained pre-cooled tank. The ratio of dichloromethane to hardening solution ratio was 1:16. This dispersion was then stirred (350 to 450 rpm) for 1 hour at 3° C. The solvent mixture was decanted and fresh heptane of 2.5:100 ratio relative to hardening solution was added at 3° C. and held for 1 hour to rinse off residual silicone oil, ethanol and dichloromethane on the microparticle surface.

(D) Microparticle Drying and Collection:

After drying/quenching, the microparticles were filtered and dried in a filter/dryer assembly, e.g. Vibrosifter (20 micron sieve). The dried microparticles were then rinsed with heptane of 2.5:100 ratio relative to hardening solution. The microparticles were then subjected to drying under vacuum with a constant purge of nitrogen gas at a controlled rate over about 5 days at temperature ranging from about 3° C. to about 38° C.

### Example: 2

### Process of Preparing the Exenatide-Loaded Microparticles

(A) Inner Water-in-Oil Emulsion Formation:

A water phase containing exenatide was prepared by dissolving exenatide (about 7.5% w/w) and sucrose (about 5.2% w/w) in water. Separately, an oil phase of polymer was prepared by dissolving PLGA polymer in dichloromethane (about 6% w/w). A water-in-oil emulsion was created by adding water phase in to the oil phase with the aid of a homogenizer or sonication or any other known method (W/O ratio of about 1:23). The resulting coarse emulsion was homogenized at approximately 10,000 rpm at ambient temperature. This resulted in an inner emulsion droplet size of less than 1 micron.

(B) Coacervate Formation:

Silicone oil was then added to the inner emulsion prepared in step A for coacervation. The preferred ratio of silicone oil to dichloromethane is 1.5:1. During the coacervation, dichloromethane from the polymer solution partitions into the silicone oil and begins to precipitate the polymer around the water phase containing exenatide, leading to microencapsulation.

(C) Microparticle Hardening/Quenching and Rinse:

Microparticles were added to a hardening solution of heptane/ethanol solvent mixture of 9:1 ratio in a contained pre-cooled tank. The ratio of dichloromethane to hardening solution ratio was 1:16. This dispersion was then stirred (350 to 450 rpm) for 1 hour at 3° C. The solvent mixture was decanted and fresh heptane of 2.5:100 ratio relative to hardening solution was added at 3° C. and held for 1 hour to rinse off residual silicone oil, ethanol and dichloromethane on the microparticle surface.

(D) Microparticle Drying and Collection:

After drying/quenching, the microparticles were filtered and dried in a filter/dryer assembly, e.g. Vibrosifter (20 micron sieve). The dried microparticles were then rinsed with heptane of 2.5:100 ratio relative to hardening solution, to remove excess silicon oil. The microparticles were then subjected to drying under vacuum with a constant purge of nitrogen gas at a controlled rate over about 5 days at temperature ranging from about 3° C. to about 38° C.

### Example: 3

### Process of Preparing the Exenatide-Loaded Microparticles According to an Embodiment of the Invention by a Modified Double-Emulsion Solvent Evaporation Method (W/O/W)

Lyophilized exenatide powder (7.5% w/w), sucrose (about 2.6% w/w) and gelatin (about 1.5% w/w) were dissolved in 0.03 M sodium acetate buffer (pH 4.5). The aqueous exenatide solution was mixed with the oil phase comprising dichloromethane containing PLGA (6% w/w), and emulsified in a homogenizer at high speed for 30 seconds. The water in oil ratio in the primary W/O emulsion was 1:20. The primary emulsion was then added to distilled water containing PVA (1%) in the weigh ratio of 1:5. The emulsification continued at 19000 rpm for 30 seconds. The formed water-in oil-in water emulsion was stirred for 4 hours at room temperature, allowing dichloromethane to evaporate. The emulsion solidified gradually as the diffusion of the solvent from the emulsion droplets into the external phase. The Nitrogen was purged on the surface of the emulsion to facilitate DCM evaporation. The resulting microparticles were washed three times in distilled water on 20 micron sieve and freeze-dried.

### Example: 4

### Process of Preparing the Exenatide-Loaded Microparticles According to an Embodiment of the Invention by a Modified Double-Emulsion Solvent Evaporation Method (W/O/W)

Lyophilized exenatide powder (7.5% w/w) and sucrose (about 5.2% w/w) were dissolved in 0.03 M sodium acetate buffer (pH 4.5). The aqueous exenatide solution was mixed with the oil phase comprising dichloromethane containing PLGA (6% w/w), and emulsified in a homogenizer at high speed for 30 seconds. The water in oil ratio in the primary W/O emulsion was 1:20. The primary emulsion was then added to distilled water containing PVA (1%) in the weigh ratio of 1:5. The emulsification continued at 19000 rpm for 30 seconds. The formed water-in oil-in water emulsion was stirred for 4 hours at room temperature, allowing dichloromethane to evaporate. The emulsion solidified gradually as the diffusion of the solvent from the emulsion droplets into the external phase. The Nitrogen was purged on the surface of the emulsion to facilitate DCM evaporation. The resulting microparticles were washed three times in distilled water on 20 micron sieve and freeze-dried.

1. A microparticulate composition for sustained release of one or more biologically active polypeptide comprising one or more biocompatible polymers, one or more natural or synthetic mucilages, one or more sugars and optionally, one or more pharmaceutical excipients.

2. A microparticulate composition for sustained release of one or more biologically active polypeptide comprising one or more biocompatible polymers and optionally, one or more pharmaceutical excipients, wherein the composition comprises more than 5% w/w of one or more sugars.

3. The microparticulate composition of claim 2, wherein the composition comprises one or more natural or synthetic mucilages.

4. The microparticulate composition of claim 1 or 3, wherein the mucilage comprises one or more of gum acacia, Irish moss, gum karaya, gum tragacanth, gum guaiac, gum xanthane, locust bean gum, casein, gelatin, collagen, albumin, globulin, fibrin, cellulose, dextrin, pectin, starch, agar, and mannan.

5. The microparticulate composition of claim 1 or 2, wherein the sugar comprises one or more monosaccharides, disaccharides, oligosaccharides, or mixtures thereof.

6. The microparticulate composition of claim 1 or 2, wherein the composition further comprises one or more corticosteroids.

7. The microparticulate composition of claim 1 or 2, wherein the biologically active polypeptide comprises exendin-4 or agonists, analogs or derivatives thereof.

8. The microparticulate composition of claim 7, wherein the exendin-4 agonist is exenatide.

9. A microparticulate composition for sustained release of exenatide consisting essentially of one or more biocompatible polymers, exenatide, gelatin, sucrose and optionally, one or more pharmaceutical excipients.

10. The microparticulate composition of claim 9, wherein the composition comprises more than 5% w/w of one or more sugars.

11. A process for preparing the microparticulate composition of claim 1 or 2, which process comprising steps of:
(1) preparing an emulsion that comprises a first phase and a second phase, the first phase comprising biologically active polypeptide, one or more natural or synthetic mucilages, one or more sugars and optionally, one or more pharmaceutical excipients, the second phase comprising one or more biodegradable polymers and one or more solvents for the polymer; and
(2) quenching and washing of the emulsion to form microparticle suspension.

12. The process of claim 11, wherein the process further comprises step of lyophilizing the microparticle suspension.

13. A method of treating a patient having Type 2 diabetes comprises of: administering the microparticulate composition of claim 1, 2 or 9.

13. The process of claim 11, wherein the process further comprises step of lyophilizing the microparticle suspension.
